(19) United States
(12) Patent Application Publication
(10) Pub. No.: US 2008/0113043 A1
(43) Pub. Date: May 15, 2008

(54) Method for producing ginseng fruit and ginseng flower stalk with high content of ginsenoside

(76) Inventor: In-Hwan Seong, Seoul (KR)

Correspondence Address: TECHNOLOGY & INTELLECTUAL PROPERTY;STRATEGIES GROUP PC (dba TIPS GROUP), P. O. BOX 1639, LOS ALTOS, CA, 94023-1639, US

(21) Appl. No.: 11/975,354

(22) Filed: Oct. 17, 2007

(30) Foreign Application Priority Data
Oct. 18, 2006 (KR) 2006-0101539
Jul. 6, 2007 (KR) 2007-0068231
Sep. 27, 2007 (KR) 2007-0097500

(51) Int. Cl. A61K 36/254; C07J 17/00; A61K 36/258

(52) U.S. Cl. 424/728; 536/6.3

(57) ABSTRACT

Disclosed is a method for producing ginseng fruits and flower stalks with high content of ginsenoside. More particularly, the present invention provides a method for producing ginseng fruits and flower stalks with high contents of ginsenoside Rb1, ginsenoside Rb2, ginsenoside Rc, ginsenoside Rd, ginsenoside Re and other ginsenoside ingredients. According to the present invention, the ginseng fruits and flower stalks have considerably increased contents of ginsenoside Re, Rd and Rb2 compared with general red ginseng roots and/or black ginseng roots. In addition, the present invention can provide ginseng fruits and flower stalks based products with high contents of, especially, ginsenoside Rb1, ginsenoside Rb2, ginsenoside Rc, ginsenoside Rd and/or ginsenoside Re by processing the fruits and flower stalks.

This application claims priority to Korean Patent Application No. 2006-0101539, filed on Oct. 18, 2006, 2007-0068231, filed on Jul. 6, 2007, 2007-0097500 filed on Sep. 27, 2007 in the Korean Intellectual Property Office, the entire contents of which are hereby incorporated by reference.

### BACKGROUND OF THE INVENTION

1. Field of the Invention

The present invention relates to methods for cultivation of ginseng fruits and ginseng flower stalks with high content of ginsenoside, more particularly, to a method for production of ginseng fruits and ginseng flower stalks with high contents of ginsenoside ingredients such as ginsenoside Rb1, ginsenoside Rb2, ginsenoside Rc, ginsenoside Rd, ginsenoside Re and others.

2. Description of the Related Art

Ginseng, which is named Panax ginseng C. A. Meyer according to the rule of Nomenclature as one of semi-shade type perennial plants and belongs to Araliaceae, is well known in a variety of medical literatures published in China and Korea to have functions and performances beneficial to human bodies.

As a result of current researches and investigations about ginseng, ginseng is found to contain plenty of ginsenoside also called ‘Saponin’ and is often applied in manufacturing health foods and/or health supplement foods recently besides traditional uses thereof such as medicines.

Ginseng cultivation usually takes at least four (4) years and, preferably, six (6) years to produce the best quality of ginseng. That is, ginseng needs a long period of time until it is completely grown and a harvest of the ginseng is usually less than an original amount thereof to be cultivated due to different reasons including varied insects and damage from disease during the cultivation, thus, leading to an increase in price and requirement of much time to cultivate ginseng fruits.

Ginseng has roots and rhizomes under the ground, and stems, leaves and fruits above the ground. More particularly, ginseng has roots including fine roots, lateral roots and main roots, rhizomes, stems continued from the rhizomes, leaves extended from the stems, flowers and fruits produced at ends of the stems, and small branches to connect the fruits and stems called flower stalks (see FIG. 1).

A ginseng plant begins to blossom in the third (3) year of growth, and generates green fruits in May which, in turn, become ripe and turn red in the middle of July and are harvested.

As ginseng growers mostly wish to obtain and utilize ginseng roots, in order to promote growth of the roots rather than reproduction growing of fruits, they generally pick and discard fruits when the ginseng plant begins to bear the fruits. In this case, they partially cut peduncles of the plant and discard them together with the fruits in ginseng beds.

Ginsenoside refers to saponin contained in the ginseng and a variety of ginsenoside ingredients are contained in the ginseng. The ginsenoside ingredients of the ginseng include, for example, Panaxadiol PD, Panaxatriol PT, Oleanane species, etc. The ginseng also contains non-ginsenoside materials including carbohydrates such as starch, anti-oxidation aromatic compounds of polyacetylene, Gomisin N-A to protect livers of human beings, acidic peptides with insulin-like functions and so on, other than the ginsenoside ingredients.

Ginsenoside has pharmacological performances principally effective to central nervous system, endocrine system, immune system, metabolic system, etc. to exhibit various effects in regard to body modulation function. Additionally, the ginsenoside is well known to have a plurality of advantages including: (1) a great lipolysis ability and promotion of nutrition absorption and digestion; (2) promotion of metabolism by activating enzymes in cells; (3) refreshment and improvement in fatigue, weakness, anorexia nervosa and the like; and (4) acceleration of serum protein synthesis, and so on.

The present inventors found and suggested that a great amount of ginsenoside Re shows central nervous inhibition, DNA and RNA behavior promotion, plasmin activation and promotion of adrenocorticotropic hormone release, Rd promotes release of adrenal cortex hormone, and Rb2 exhibits central nervous inhibition, DNA and RNA behavior promotion, plasmin activation, promotion of adrenocorticotropic hormone release and anti-diabetic effect.

However, such ginseng fruits and ginseng flower stalks are substantially discarded as waste after removal thereof from the ginseng plant, and there is a lack of use of ginsenoside ingredients contained in them.

### SUMMARY OF THE INVENTION

Accordingly, the present invention is directed to solve the problems as described above and, an object of the present invention is to provide a method for production of ginseng fruits and flower stalks with high contents of ginsenoside ingredients, for example, ginsenoside Rb1, ginsenoside Rb2, ginsenoside Rc, ginsenoside Rd, ginsenoside Re and others.

Another object of the present invention is to provide a method for producing ginseng fruits and flower stalks with high content of ginsenoside by means of: treating the ginseng fruits and flower stalks by light irradiation; steaming the treated fruits and flower stalks with water vapor then drying the steamed product at least one time; and precipitating the dried product in a solution containing ginsenoside decomposers.

In order to accomplish the above objects, there is provided a method for producing ginseng fruits and flower stalks with high content of ginsenoside, comprising the steps of: preparing the ginseng fruits and flower stalks by light irradiation; steaming the treated fruits and flower stalks with water vapor then drying the steamed product at least one time; and precipitating the dried product in a solution containing ginsenoside decomposers.

According to the present invention, the produced ginseng fruits and flower stalks contain abundant ginsenoside ingredients which are appropriately added to health supplement foods and/or used to manufacture ginsenoside-rich health supplement foods.

The ginseng fruits and flower stalks with high content of ginsenoside according to the present invention are obtained from the ginseng fruits and flower stalks discarded in farm fields and processed into ginsenoside-rich ginseng products.

The ginseng fruits and flower stalks of the present invention may undergo a concentration process using a concentrator to produce a ginsenoside enriched extract such as ginsenoside Rb1, ginsenoside Rb2, ginsenoside Rc, ginsenoside Rd, ginsenoside Re, etc.

Accordingly, the present invention can use ginseng fruits to produce the ginseng fruits and flower stalks with high contents ginsenoside Rb1, ginsenoside Rb2, ginsenoside Rc, ginsenoside Rd, ginsenoside Re and other ginsenoside ingredients besides traditionally used ginseng roots, therefore, can contribute to an increase in income of the ginseng farmers.

### BRIEF DESCRIPTION OF THE DRAWINGS

These and other objects, features, aspects, and advantages of preferred embodiments of the present invention will be more fully described in the following detailed description, taken in conjunction with the accompanying drawing. In the drawing:

FIG. 1 illustrates construction in details of fruits, leaves, stems and roots of a ginseng plant.

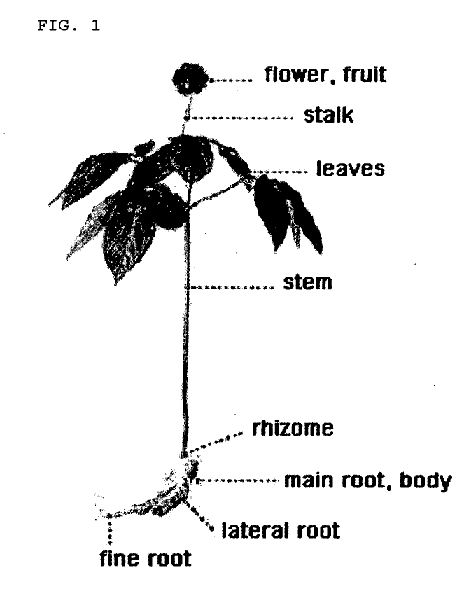

### DETAILED DESCRIPTION OF THE INVENTION

The present invention provides a method for producing ginseng fruits and flower stalks with high content of ginsenoside, comprising the steps of: treating the ginseng fruits and flower stalks by light irradiation; steaming the treated fruits and flower stalks with water vapor then drying the steamed product at least one time; and precipitating the dried product in a solution containing desirable ginsenoside decomposers.

Hereafter, the present invention will be described in detail, especially, with regard to each of the steps for production of the ginseng fruits and flower stalks with high content of ginsenoside.

1. Steaming and drying the ginseng fruits and flower stalks after light irradiation.

In order to increase contents of the ginsenoside ingredients in the fruits and flower stalks, they are preferably subjected to light irradiation after gathering the fruits and flower stalks from ginseng farm fields.

Such light irradiation can use sun light containing all of ultraviolet (UV) rays, visible rays and infrared rays.

Preferably, such light irradiation can use at least one selected from UV rays, visible rays and infrared rays.

UV rays used in the present invention preferably comprise UV rays at 10 to 397 nm wavelength.

The visible rays used in the present invention preferably comprise visible rays at 380 to 770 nm wavelength.

The infrared rays used in the present invention preferably comprise infrared rays at 0.75 μm to 1,000 μm wavelength.

The light irradiation to the ginseng fruits and flower stalks is conducted for about 1 to 24 hours. When the irradiation time is less than 1 hour, the content of ginsenoside in the ginseng fruits and flower stalks is only slightly increased. On the other hand, if the irradiation time exceeds 24 hours, there is not a remarkably improved effect based on increase in the ginsenoside content and, conversely, there is a possibility of reducing other nutritive substances contained in the fruits and flower stalks. Therefore, the light irradiation is preferably performed for 1 to 24 hours for producing the ginseng fruits and flower stalks with high content of ginsenoside of the present invention.

In order to increase the ginsenoside content and/or obtain a variety of ginsenoside ingredients during the production of the ginseng fruits and flower stalks with high content of ginsenoside of the present invention, the ginseng fruits and flower stalks may be treated by a steaming process.

The steaming process is conducted by using water vapor at 90 to 120° C. several times for 1 to 3 hours per time.

The steaming process of the ginseng fruits and flower stalks is preferably conducted at least one time.

More preferably, the steaming process of the ginseng fruits and flower stalks is conducted by using water vapor at 90 to 120° C. four (4) times for 1 to 3 hours per time.

The steamed fruits and flower stalks are dried to remove the water vapor.

In case of applying the steaming process several times, the drying process is usually carried out instantly after completing each of the steaming processes, followed by further steaming and drying processes.

The drying process after the steaming process for the fruits and flower stalks is carried out at 65 to 75° C. until the water vapor remained on the outer surface of the fruits and flower stalks is dried out.

The drying process for the steamed product of the fruits and flower stalks may be carried out using general methods commonly known in the art. One example of these method may include any one selected form air drying at ordinary temperature, heat drying with hot air, freeze drying at low temperature and so on. For the present invention, the drying process for the fruits and flower stalks is preferably carried out at 65 to 75° C. until the water vapor remained on the outer surface of the fruits and flower stalks is dried out.

2. Precipitation of ginseng fruits in a solution containing a desirable ginsenoside decomposer, after the steaming and drying processes.

The ginseng contains various kinds of ginsenoside ingredients, which are not directly absorbed in a human body, but transferred into substances capable of being absorbed in the human body by the decomposer such as body enzymes for decomposition of the ginsenoside before the absorption thereof.

Accordingly, the present invention can accomplish the production of ginseng fruits and flower stalks with high content of ginsenoside and/or ginsenoside ingredients easily absorbed in the human body, by the steps of: treating the ginseng fruits and flower stalks by light irradiation; steaming the treated fruits and flower stalks with water vapor then drying the steamed product; and precipitating the dried product in a solution containing the ginsenoside decomposer.

The ginsenoside decomposer may include any one selected form Hasegawa bacteria, α-glucosidase, α-Rhamnosidase, soyasapogenol B and the like, for example.

The solution containing the ginsenoside decomposer is prepared by adding the ginsenoside decomposer to a pure water in an amount of 1 to 20% by weight (abbrev. to “wt. %”) to total weight of the pure water.

If the amount is less than 1 wt. % to total weight of the pure water, the content of ginsenoside and/or ginsenoside ingredients easily absorbed in the human body is not increased. On the other hand, when the amount exceeds 20 wt. %, there is no remarkably beneficial effect in terms of increase in the content of ginsenoside and/or ginsenoside ingredients easily absorbed in the human body, and there may be adverse effects to other ingredients contained in the ginseng fruits and flower stalks and is thus not preferable. Therefore, the ginsenoside decomposer is preferably added in the amount of 1 to 20 wt. % to total weight of the pure water to prepare the solution, in which the ginseng fruits and flower stalks are precipitated after the light irradiation and the water vapor steaming and drying processes.

The fruits and flower stalks treated by the light irradiation and the water vapor steaming and drying processes may be subjected to the precipitation process using the ginsenoside decomposer for 10 minutes to 24 hours.

The present invention adopts at least one additional treatment selected from ultrasonic treatment, low frequency treatment, electric field treatment and magnetic field treatment after any one of the above steps, in order to produce the ginseng fruits and flower stalks containing plenty of ginsenoside ingredients.

The ultrasonic treatment is performed at not less than 20 kHz for more than 10 minutes.

Preferably, the ultrasonic treatment is performed at 20 to 50 kHz for 10 minutes to 2 hours.

According to the present inventive method for producing the ginseng fruits and flower stalks with high content of ginsenoside, as a result of applying various conditions for the ultrasonic treatment, the ultrasonic treatment under the above condition was demonstrated to provide the fruits and flower stalks with high contents of ginsenoside ingredients satisfying the purpose of the present invention.

The low frequency treatment is performed at not more than 10 kHz for more than 30 minutes.

Preferably, the low frequency treatment is performed at 5 to 10 kHz for 30 minutes to 2 hours.

According to the present inventive method for producing the ginseng fruits and flower stalks with high content of ginsenoside, as a result of applying various conditions for the low frequency treatment, the low frequency treatment under the above condition was demonstrated to provide the fruits and flower stalks with high contents of ginsenoside ingredients satisfying the purpose of the present invention.

The electric field treatment is performed in an electric field of not less than 5 kv for more than 30 seconds.

Preferably, the electric field treatment is performed at 5 to 20 kv for 30 seconds to 5 minutes.

According to the present inventive method for producing the ginseng fruits and flower stalks with high content of ginsenoside, as a result of applying various conditions for the electric field treatment, the electric field treatment under the above condition was demonstrated to provide the fruits and flower stalks with high contents of ginsenoside ingredients satisfying the purpose of the present invention.

The magnetic field treatment is performed in a magnetic field of not less than 5 Gauss (G) for more than 30 minutes.

Preferably, the magnetic field treatment is performed at 5 to 30 G for 30 minutes to 1 hour.

According to the present inventive method for producing the ginseng fruits and flower stalks with high content of ginsenoside, as a result of applying various conditions for the magnetic field treatment, the magnetic field treatment under the above condition was demonstrated to provide the fruits and flower stalks with high contents of ginsenoside ingredients satisfying the purpose of the present invention.

The present invention includes the ginseng fruits processed by the above method.

The present invention includes the ginseng flower stalks processed by the above method.

The present invention includes health supplement foods which contain the ginseng fruits with high content of ginsenoside processed by the above method.

The present invention includes health supplement foods which contain the ginseng flower stalks with high content of ginsenoside processed by the above method.

The above health supplement foods may be provided in forms of, for example, liquid drinks, solid tablets, pills, powders, capsules and the like.

As one of constitutional components of a health supplement food according to the present invention, the ginseng fruits and/or flower stalks with high content of ginsenoside are preferably contained in an amount of 0.1 to 10 wt. % to total weight of the health supplement food. The health supplement food may further include additional substances such as, for example, fillers, lubricants, binders, disintegrants, etc. Such additional substances are added to the health supplement foods in forms of, for example, solid tablets, pills, powders, capsules and the like. Since these substances are commonly used and are appropriately selected by those skilled in the related arts, the present invention does not include detailed description thereof.

The present inventive ginseng fruits and/or flower stalks contained in the health supplement foods preferably have at least one selected from various forms including powders, juices, hot water extracts and organic solvent extracts and so on.

The ginseng fruits and/or flower stalks in the powder form are obtained by using a grinder or miller to pulverize the fruits and/or flower stalks.

The ginseng fruits and/or flower stalks in the juice form are obtained by using a screw juicer or a juice extractor to press out the extract of the fruits and/or flower stalks.

The ginseng fruits and/or flower stalks in the hot water extract form are obtained by introducing crude fruits and/or flower stalks in the pure water in an amount of 3 to 10 times the fruits and/or flower stalks, treating the mixture at 80 to 120° C. until the amount of the pure water is reduced to 10 to 50% of the original amount thereof to form an extract, filtering the mixture to obtain a filtrate containing the extract and/or concentrating the filtrate to obtain the concentrated product.

The ginseng fruits and/or flower stalks in the organic solvent extract form are obtained by introducing crude fruits and/or flower stalks in the organic solvent in an amount of 2 to 5 times the fruits and/or flower stalks, treating the mixture at 65 to 75° C. for 1 to 24 hours to form an extract, filtering the mixture to obtain a filtrate containing the extract and/or concentrating the filtrate to obtain the concentrated product. The organic solvent can be removed from the filtrate through a further removal step for the organic solvent.

The organic solvent used to produce the organic solvent extract of the ginseng fruits and/or flower stalks according to the present invention preferably includes alcohol solvents in which number of carbon atoms ranges from 1 to 20.

The organic solvent used to produce the organic solvent extract of the ginseng fruits and/or flower stalks according to the present invention preferably includes, for example, methanol, ethanol, propanol, butanol and pentanol.

The organic solvent used to produce the organic solvent extract of the ginseng fruits and/or flower stalks according to the present invention is more preferably ethanol.

Hereinafter, the present invention will be described in more detail in the following examples with reference to the accompanying drawing. However, these are intended to illustrate the invention as preferred embodiments of the present invention and do not limit the scope of the present invention.

### EXAMPLE 1-1

The following process is used to produce the ginseng fruits with high contents of ginsenoside ingredients.

Ginseng fruits gathered from ginseng plants underwent infrared irradiation using infrared rays at 100 μm wavelength for 10 hours. 1 kg of the treated fruits with infrared rays were steamed using the water vapor at 95° C. for 1 hour and dried at 70° C.

After such steaming and drying process were repeated four times, 1 kg of the processed fruits were added in a solution containing a ginsenoside decomposer and precipitated for 30 minutes in the solution.

The solution containing the ginsenoside decomposer was a solution prepared by adding α-glucosidase as the ginsenoside decomposer in an amount of 10 wt. % to total weight of pure water.

### EXAMPLE 1-2

The ginseng flower stalks with high contents of ginsenoside ingredients were produced in the same manner as in Example 1-1, except that flower stalks gathered from the ginseng plants were used.

### EXAMPLE 2-1

The ginseng fruits with high contents of ginsenoside ingredients were produced in the same manner as in Example 1-1, except that the fruits were subjected to UV irradiation using UV rays at 100 nm wavelength for 5 hours.

### EXAMPLE 2-2

The ginseng flower stalks with high contents of ginsenoside ingredients were produced in the same manner as in Example 2-1, except that flower stalks gathered from the ginseng plants were used.

### EXAMPLE 3-1

The ginseng fruits with high contents of ginsenoside ingredients were produced in the same manner as in Example 1-1, except that the fruits were subjected to visible ray irradiation using visible rays at 500 nm wavelength for 7 hours.

### EXAMPLE 3-2

The ginseng flower stalks with high contents of ginsenoside ingredients were produced in the same manner as in Example 3-1, except that flower stalks gathered from the ginseng plants were used.

### EXAMPLE 4-1

Ginseng fruits gathered from ginseng plants underwent infrared irradiation using infrared rays at 100 μm wavelength for 10 hours.

1 kg of the treated fruits with infrared rays were steamed using the water vapor at 95° C. for 1 hour and dried at 70° C.

After such steaming and drying process were repeated four times, 1 kg of the processed fruits were added in a solution containing a ginsenoside decomposer and precipitated for 30 minutes. 1 kg of the precipitated fruits underwent ultrasonic treatment at 40 kHz for 1 hour.

The solution containing the ginsenoside decomposer was a solution prepared by adding α-glucosidase as the ginsenoside decomposer in an amount of 10 wt. % to total weight of pure water.

### EXAMPLE 4-2

The ginseng flower stalks with high contents of ginsenoside ingredients were produced in the same manner as in Example 4-1, except that flower stalks gathered from the ginseng plants were used.

### EXAMPLE 5-1

The ginseng fruits with high contents of ginsenoside ingredients were produced in the same manner as in Example 4-1, except that the fruits were subjected to low frequency treatment at 7 kHz for 1 hour instead of the ultrasonic treatment.

### EXAMPLE 5-2

The ginseng flower stalks with high contents of ginsenoside ingredients were produced in the same manner as in Example 5-1, except that flower stalks gathered from the ginseng plants were used.

### EXAMPLE 6-1

The ginseng fruits with high contents of ginsenoside ingredients were produced in the same manner as in Example 4-1, except that the fruits were subjected to electric field treatment at 15 kv for 3 minutes instead of the ultrasonic treatment.

### EXAMPLE 6-2

The ginseng flower stalks with high contents of ginsenoside ingredients were produced in the same manner as in Example 6-1, except that flower stalks gathered from the ginseng plants were used.

### EXAMPLE 7-1

The ginseng fruits with high contents of ginsenoside ingredients were produced in the same manner as in Example 4-1, except that the fruits were subjected to magnetic field treatment at 20 Gauss for 40 minutes instead of the ultrasonic treatment.

### EXAMPLE 7-2

The ginseng flower stalks with high contents of ginsenoside ingredients were produced in the same manner as in Example 7-1, except that flower stalks gathered from the ginseng plants were used.

### EXPERIMENTAL EXAMPLE 1

Each of the ginseng fruits produced according to Examples 1-1, 2-1, 3-1, 4-1, 5-1, 6-1 and 7-1 was introduced in the pure water of five times total weight of the fruits, followed by hot water extraction at 100° C. until the amount of the pure water was reduced to 30% of the original amount thereof to form a ginseng extract. The resulting extracts from the fruits according to the above examples, respectively, were used as test samples.

Ginseng roots commercially available in markets were treated in the same manner and condition for the hot water extraction as described above. The resulting extract was used as a test control.

The test samples and the test control were subjected to measurement of ginsenoside content relative to the ginseng extract and the results are shown in the following Table 1.

TABLE 1
Content of ginsenoside in ginseng extract according to each of
Examples 1-1, 2-1, 3-1, 4-1, 5-1, 6-1 and 7-1 and the control

| Examples | Content of ginsenoside |
|---|---|
| Example 1-1 | 105.4 |
| Example 2-1 | 105.2 |
| Example 3-1 | 105.5 |
| Example 4-1 | 107.6 |
| Example 5-1 | 107.5 |
| Example 6-1 | 107.4 |
| Example 7-1 | 107.6 |
| Control | 100 |

Measured values of the ginsenoside content were determined with reference to Rd as a standard by phase separating the sample into water and butanol, drying the butanol phase, dissolving the dried sample and the standard Rd in glacial acetic acid, adding a 15% antimony trichloride solution to the prepared solution, heating the mixture at 60° C. for 30 minutes, cooling the heated solution and determining absorbance at 525 nm. Herein, the content of ginsenoside in the control was defined to 100 and the content of ginsenoside in each of the test samples according to the above examples was determined as a relative ratio to the content of the control. As the measured value is higher, the content of ginsenoside is increased.

### EXPERIMENTAL EXAMPLE 2

Each of the ginseng flower stalks produced according to Examples 1-2, 2-2, 3-2, 4-2, 5-2, 6-2 and 7-2 was introduced in the pure water of five times total weight of the flower stalks, followed by hot water extraction at 100° C. until the amount of the pure water was reduced to 25% of the original amount thereof to form a ginseng extract. The resulting extracts from the flower stalks according to the above examples, respectively, were used as test samples.

Ginseng flower stalks gathered in ginseng farm fields were treated in the same manner and condition for the hot water extraction as described above. The resulting extract was used as a test control.

The test samples and the test control were subjected to measurement of ginsenoside content in the same manner as in Experimental Example 1, and the results are shown in the following Table 2.

TABLE 2
Content of ginsenoside in ginseng extract according to each of
Examples 1-2, 2-2, 3-3, 4-2, 5-2, 6-2, 7-2 and the control

| Examples | Content of ginsenoside |
|---|---|
| Example 1-2 | 104.2 |
| Example 2-2 | 104.1 |
| Example 3-2 | 104.3 |
| Example 4-2 | 106.4 |
| Example 5-2 | 106.3 |
| Example 6-2 | 106.2 |
| Example 7-2 | 106.4 |
| Control | 100 |

### EXPERIMENTAL EXAMPLE 3

The present inventors asked Life Sciences and Chemical Analysis Center of Joongbu University in Korea to analyze ginsenoside content in the ginseng fruits produced according to Example 1-1 of the present invention, compared with the ginsenoside content in each of black ginseng roots, black ginseng fine roots, red ginseng roots and red ginseng fine roots, all of which were obtained according to Korean Patent No. 10-0543862. The analysis results are shown in the following Table 3.

TABLE 3
Analysis results for contents of ginsenoside ingredients in
ginseng fruits, compared with conventional ginseng products

| | | | Black | | Red |
|---|---|---|---|---|---|
| | | Black | ginseng | Red | ginseng |
| | Example | ginseng | fine | ginseng | fine |
| Species | 1-1 | roots | roots | roots | roots |
| Ginsenoside Rg3 | 4.51 | 9.18 | 14.23 | 0.18 | 0.10 |
| Ginsenoside Rb1 | 12.98 | 1.78 | 4.78 | 0.69 | 1.37 |
| Ginsenoside Rb2 | 23.11 | 1.51 | 3.99 | 0.19 | 0.71 |
| Ginsenoside Rc | 14.74 | 1.32 | 3.93 | 0.25 | 0.12 |
| Ginsenoside Rd | 31.05 | 1.14 | 5.68 | 0.26 | 0.10 |
| Ginsenoside Re | 135.16 | 0.20 | 1.40 | 0.40 | 0.99 |
| Ginsenoside Rf | — | 0.68 | 1.00 | 0.22 | 0.31 |
| Ginsenoside Rg2 | 3.01 | 0.28 | 0.54 | 0.18 | 0.22 |
| Ginsenoside Rh1 | 2.22 | 1.06 | 1.17 | confirmed | confirmed |
| Ginsenoside Rh2 | 0.04 | — | — | — | — |
| Ginsenoside X | 0.52 | — | — | — | — |

—: not detected

As shown in the result of the Table 3, it was identified that the contents of ginsenoside ingredients in the ginseng fruits produced according to Example 1-1 of the present invention and, especially, the contents of ginsenoside ginsenoside Rb1, ginsenoside Rb2, ginsenoside Rc, ginsenoside Rd, and ginsenoside Re are considerably increased, compared with the contents of ginsenoside ingredients in each of black ginseng roots, black ginseng fine roots, red ginseng roots and red ginseng fine roots which were obtained according to Korean Patent No. 10-0543862.

While the present invention has been described with reference to the preferred embodiments, examples and experimental examples, it will be understood by those skilled in the art that various modifications and variations may be made therein without departing from the scope of the present invention as defined by the appended claims.

Consequently, the present invention can produce the ginseng fruits and flower stalks with high content of ginsenoside from ginseng fruits and flower stalks containing abundant saponin besides traditionally used ginseng roots, thereby manufacturing the ginseng fruits and flower stalks with high content of ginsenoside themselves, and commercially processed products thereof and contributing to an increase in income of ginseng farmers.

What is claimed is:

1. A method for producing ginseng fruits and flower stalks with high content of ginsenoside, comprising the steps of: treating the ginseng fruits and flower stalks by light irradiation; steaming the treated fruits and flower stalks with water vapor then drying the steamed product at least one time; and precipitating the dried product in a solution containing a ginsenoside decomposer.

2. The method according to claim 1, wherein the light is selected from infrared rays, visible rays and ultraviolet (UV) rays.

3. The method according to claim 1, wherein the steamed and dried ginseng fruits and flower stalks are precipitated in the solution containing at least one selected from Hasegawa bacteria, α-glucosidase, α-Rhamnosidase and soyasapogenol B as the ginsenoside decomposer.

4. The method according to claim 1, further comprising a treatment process of the ginseng fruits and flower stalks by at least one selected from ultrasonic treatment, low frequency treatment, electric field treatment and magnetic field treatment after the precipitation step.
